(19) United States
(12) Patent Application Publication
(10) Pub. No.: US 2017/0181689 A1
(43) Pub. Date: Jun. 29, 2017

(54) System and Method for Measuring the Muscle Tone

(71) Applicants: METAL INDUSTRIES RESEARCH & DEVELOPMENT CENTRE, Kaohsiung City (TW); KAOHSIUNG VETERANS GENERAL HOSPITAL, Kaohsiung City (TW)

(72) Inventors: Ko-Lung Lin, Kaohsiung City (TW); Ming-Hui CHENG, Kaohsiung City (TW); Chih-Ming Kung, Kaohsiung (TW); Ho-Chung Fu, Kaohsiung City (TW); Yu-Min TING, Kaohsiung City (TW)

(21) Appl. No.: 14/980,272

(22) Filed: Dec. 28, 2015

(51) Int. Cl. A61B 5/00; A61B 5/11

(52) U.S. Cl. CPC A61B 5/4519; A61B 5/7267; A61B 5/1122; A61B 5/1123; A61B 5/7278; A61B 2560/0252; A61B 2562/0247; A61B 2562/0219; A61B 2562/0223

(57) ABSTRACT

A method for measuring a muscle tone is used in a muscle tone measuring system including a sensing end and a processing end. The method for measuring the muscle tone includes respectively arranging the sensing end and the processing end on first and second limb parts of a limb that are interconnected by a joint of the limb. The sensing end detects a strength of a force that is applied to the first limb part and a plurality of physical quantities of a movement of the first limb part. The processing end detects an environment temperature and the physical quantities of a movement of the second limb part, and collects the physical quantities of the movements of the first and second limb parts, the environment temperature and the strength of the force according to a sampling rate during a period of measuring time. The muscle tone measuring system is also provided.

### BACKGROUND OF THE INVENTION

1. Field of the Invention

The present disclosure generally relates to a method and a system for measuring the muscle tone and, more particularly, to a method and a system that can assist the medical staff in measuring the level of dystonia of a rehabilitant.

2. Description of the Related Art

As the advance of the medical technology, the probability of successful treatment of diseases has been increased, leading to an increased average life expectancy. However, some patients are not completely recovered from the disease after the therapy. For example, the patients with brain or spinal cord injury will require further rehabilitation if they still suffer from hemiplegia after the therapy. Thus, due to the coming of the aging society, the medical demand is going to be huge.

The patients with apoplectic hemiplegia, cerebral palsy, parkinson's disease or spinal cord injury often experience dystonia during the rehabilitation process and will exhibit some clinical symptoms such as muscle spasticity and rigidity. During the rehabilitation process, the doctor needs to apply a force to the limbs of the patient and determines whether there is an abnormality in the muscle tone based on the related rating scale and personal experience. Different doctors may have different diagnostic results, and therefore the diagnosis is not objective.

Furthermore, the conventional measuring method often requires an instrument to measure the quantitative data of the movement of the limb, such as an isokinetic dynamometer or a miniature muscle strength detector. As the limb includes two limb parts interconnected by a joint, the changes of the angle and tone of each limb part during the flection of the limb cannot be obtained simply from the data of the limb. As a result, the doctor (or therapist) can have an incorrect diagnosis. In addition, the conventional isokinetic dynamometer is mainly designed for athletes, and is expensive and has a large volume. Moreover, the isokinetic dynamometer can only perform a simple muscle strength test but cannot measure the level of abnormality of the muscle. As such, the doctor or the therapist is not able to evaluate the condition of the patient and cannot promptly handle with the situation.

In light of this, it is necessary to improve conventional instrument and measuring method.

### SUMMARY OF THE INVENTION

It is therefore the objective of this disclosure to provide a method for measuring the muscle tone of a patient, so as to measure the level of dystonia of the patient.

It is another objective of this disclosure to provide a system for measuring the muscle tone of a patient, so as to measure the level of dystonia of the patient.

In an embodiment of the disclosure, a system for measuring a muscle tone is disclosed. The system may include a sensing end and a processing end. The sensing end is arranged on a first limb part of a limb and includes a pressure sensor and a first inertia detector. The pressure sensor detects a strength of a force that is applied to the first limb part of the limb. The first inertia detector measures a plurality of physical quantities of a movement of the first limb part of the limb. The processing end is arranged on a second limb part of the limb. The first and second limb parts are interconnected by a joint of the limb. The processing end includes a temperature sensor, a second inertia detector and a processor. The temperature sensor detects an environment temperature. The second inertia detector measures a plurality of physical quantities of a movement of the second limb part of the limb. The processor is electrically connected to the second inertia detector, the temperature sensor, the pressure sensor and the first inertia detector. The processing end collects output signals of the temperature sensor, the pressure sensor, the first inertia detector and the second inertia detector according to a sampling rate during a period of measuring time.

In another embodiment, a method for measuring a muscle tone for use in the above system is disclosed. The muscle tone measuring system includes a sensing end and a processing end. The processing end is electrically connected to the sensing end and coupled with a mobile calculation device. The method for measuring the muscle tone includes arranging the sensing end on a first limb part of a limb and arranging the processing end on a second limb part of the limb. The first and second limb parts are interconnected by a joint of the limb. The sensing end is able to detect a strength of a force that is applied to the first limb part of the limb and a plurality of physical quantities of a movement of the first limb part of the limb. The processing end is able to detect an environment temperature and a plurality of physical quantities of a movement of the second limb part, and to collect the plurality of physical quantities of the movement of the first limb part, the plurality of physical quantities of the movement of the second limb, the environment temperature and the strength of the force according to a sampling rate during a period of measuring time.

In a form shown, the processing end further includes a communication unit electrically connected to the processor and coupled with a mobile calculation device. The processing end generates a discrete-time data from the output signals of the temperature sensor, the pressure sensor, the first inertia detector and the second inertia detector, and generates a spasticity level value according to the discrete-time data and two trained weighting matrixes through the use of a machine learning method. The machine learning method is artificial neural network or support vector machine. The processing end calculates an angle value and a velocity value corresponding to a motion of the joint according to the output signals of the first and second inertia detectors, and generates the spasticity level value according to the two trained weighting matrixes and the environment temperature, the strength of the force, the angle value and the velocity value in each of a plurality of time points. Thus, the level of dystonia can be determined based on the above signals, and the predicted result is reliable in determining the muscle tone condition of the patient. As a result, the method and system for measuring the muscle tone according to the disclosure are able to help the doctors during the diagnosis, thus reducing the probability of misdiagnosis.

In the form shown, each of the first and second inertia detectors includes a three-axis accelerometer, a three-axis gyroscope and a three-axis magnetometer. The processing end includes a display electrically connected to the processor. Since the system does not consist of expensive components, the manufacturing cost is low.

The system and method for measuring the muscle tone are able to automatically generate the spasticity level value during the testing procedure of the patient. The value of the spasticity level is predicted according to the weighting matrixes that have been trained using a significant amount of training data. Thus, the accuracy of the prediction is high, and the predicted result is reliable in determining the muscle tone condition of the patient. As a result, the probability of misdiagnosis resulting from subjective judgment of the doctor is reduced. Since the system does not consist of expensive components, the manufacturing cost is low. Furthermore, the system is easy to operate so that it can be widely used in home care or medical care, increasing the objectivity of the predicted result and avoiding the misdiagnosis resulting from subjective judgment of the doctor.

### BRIEF DESCRIPTION OF THE DRAWINGS

The present disclosure will become more fully understood from the detailed description given hereinafter and the accompanying drawings which are given by way of illustration only, and thus are not limitative of the present disclosure, and wherein:

FIG. 1 shows a block diagram of a system for measuring the muscle tone according to a first embodiment of the disclosure.

FIG. 2 shows a sensing end and a processing end of the system for measuring the muscle tone according to the first embodiment of the disclosure.

FIG. 3 shows the use of the system for measuring the muscle tone according to the first embodiment of the disclosure.

FIG. 4 shows a flowchart of a method for measuring the muscle tone according to a second embodiment of the disclosure.

FIG. 5 shows the learning, training and prediction of the machine according to the disclosure.

In the various figures of the drawings, the same numerals designate the same or similar parts. Furthermore, when the terms “first”, “second”, “third”, “fourth”, “inner”, “outer”, “top”, “bottom”, “front”, “rear” and similar terms are used hereinafter, it should be understood that these terms have reference only to the structure shown in the drawings as it would appear to a person viewing the drawings, and are utilized only to facilitate describing the disclosure.

### DETAILED DESCRIPTION OF THE INVENTION

The term “couple/coupled” used hereinafter refers to the electromagnetic coupling technology which provides a communication mechanism between two electronic devices. The electromagnetic coupling technology may be, but not limited to, Bluetooth, Wi-Fi or 4G-LTE which are used for wireless data transmission, as it can be readily appreciated by the persons having ordinary skill in the art.

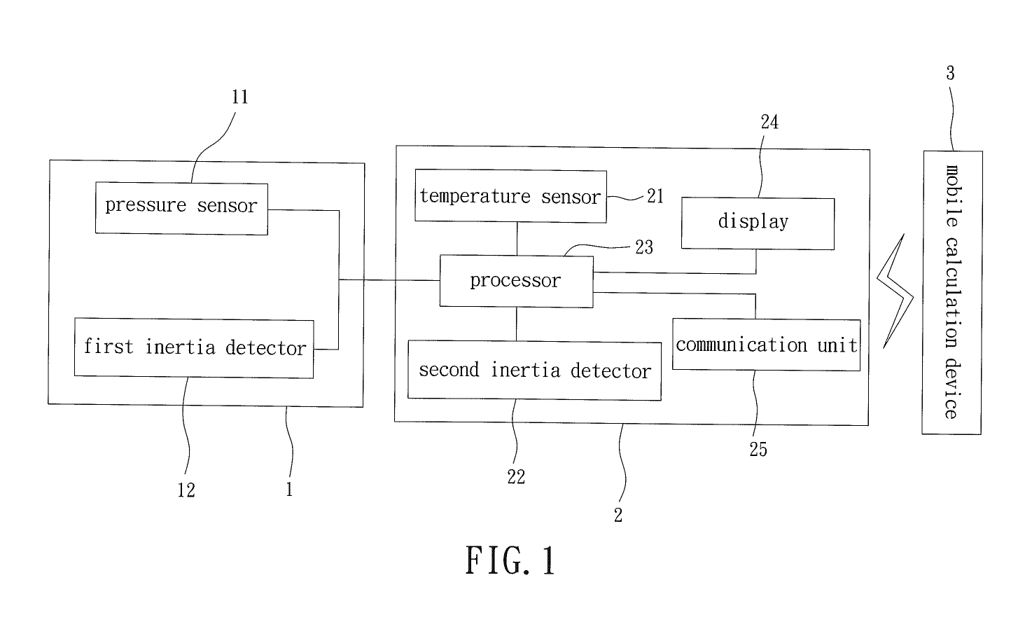

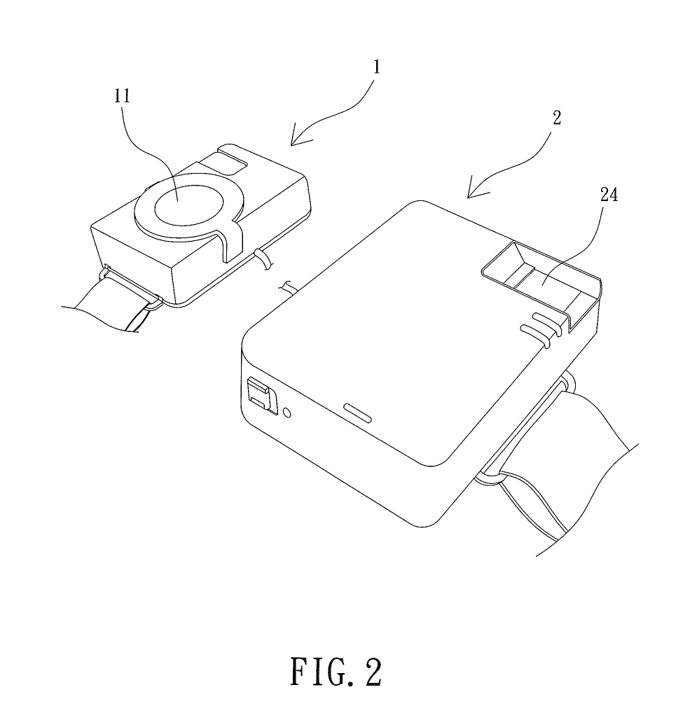

FIG. 1 shows a block diagram of a system for measuring the muscle tone according to a first embodiment of the disclosure. The system may include a sensing end 1 and a processing end 2 electrically connected to the sensing end 1. The sensing end 1 and the processing end 2 may be seen in FIG. 2. As the limb includes two limb parts (first and second limb parts) interconnected by a joint, the sensing end 1 and the processing end 2 are used to detect the information regarding the applied force and the movement of the first and second limb parts, respectively. The obtained information may be used to determine a level of dystonia of the patient.

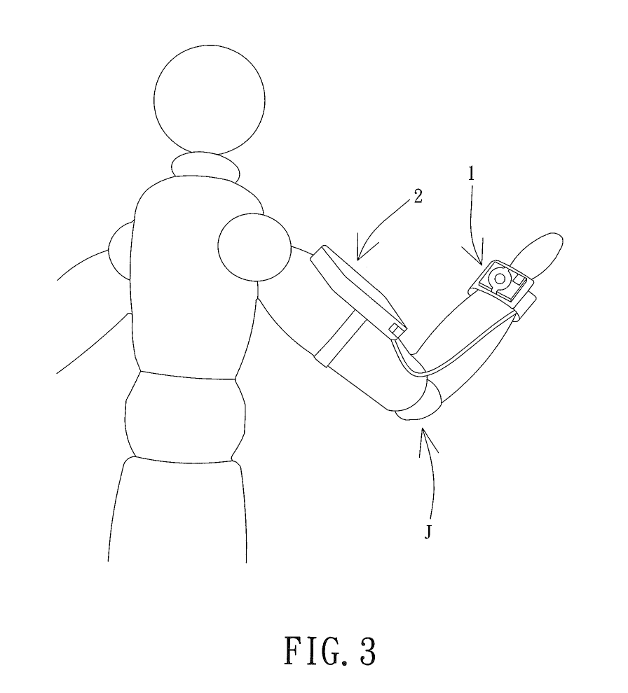

Referring to FIG. 1 again, the sensing end 1 may include a device capable of detecting the information regarding the temperature, the pressure and the movement range. The sensing end 1 is worn on the first limb part of the limb (which is connected to the second limb part of the limb via a joint such as a wrist joint, an elbow joint, a knee joint or an ankle joint) and detects the information regarding the environment temperature, the force that is applied to the first limb part, and the physical quantities of the movement of the first limb part. In FIG. 3, the sensing end 1 is worn on the wrist (first limb part) which is connected to an arm via an elbow joint “J.” In the first embodiment, the sensing end 1 may include a pressure sensor 11 and a first inertia detector 12. The pressure sensor 11 may be a piezoeletric sensor that detects the strength of the force that is applied to the first limb part by a therapist or a doctor. The first inertia detector 12 may include a three-axis accelerometer, a three-axis gyroscope and a three-axis magnetometer, which are used to measure the physical quantities of the movement of the first limb part. The physical quantities include 3-dimensional vector information such as an acceleration, a magnetic gravity angle and a magnetic strength, but are not limited thereto.

Referring to FIG. 1 again, the processing end 2 may include a device with a temperature detection function, a movement range detection function, a data processing function and a communication function. The processing end 2 is worn on the second limb part of the limb (such as the arm which is connected to the first limb part of the limb via the elbow joint “J” as shown in FIG. 3) and detects the environment temperature and the physical quantities of the movement of the second limb part. Then, during a period of measuring time, the processing end 2 collects the information regarding the movement ranges of the first and second limb parts, the temperature and the strength of the applied force according to a sampling rate, thereby generating a discrete-time data that will be used to determine the level of dystonia of the patient. In the first embodiment, the processing end 2 may include a temperature sensor 21, a second inertia detector 22 and a processor 23. The temperature sensor 21 may be a temperature sensor with positive or negative temperature coefficient, which detects the environment temperature. However, the temperature sensor 21 may also be arranged on the sensing end 1. The second inertia detector 22 may include a three-axis accelerometer, a three-axis gyroscope and a three-axis magnetometer, which are used to measure the physical quantities of the movement of the second limb part. The physical quantities include 3-dimensional vector information such as an acceleration, a magnetic gravity angle and a magnetic strength, but are not limited thereto. The processor 23 may be a microprocessor or a digital signal processor, and is electrically connected to the temperature sensor 21, the second inertia detector 22, the pressure sensor 11 and the first inertia detector 12. The processor 23 may execute a program and collect the output signals of the pressure sensor 11, the first inertia detector 12, the temperature sensor 21 and the second inertia detector 22 according to the sampling rate (such as 1-40 times/sec) during the period of measuring time (such as 10-60 sec). For instance, the processor 23 may collect the information regarding the temperature, the strength of the applied force, the accelerations, the magnetic gravity angles and the directions in each of the 1^{st}, 2^{nd}, . . . , and n^{th} seconds. As such, the discrete-time data can be generated. However, the type of the collected information is not limited to the above.

Referring to FIGS. 1 and 2 again, the processing end 2 may further include a display 24, such as a seven-segment display or a liquid crystal display. The display 24 is electrically connected to the processor 23 to display the values of the processed parameters, such as the period of measuring time, the sampling rate, the strength value of the applied force of the pressure sensor 11, the temperature value of the temperature sensor 21, and the movement ranges of the first inertia detector 12 and the second inertia detector 22. However, this does not thus limit the disclosure.

Referring to FIG. 1 again, the system for measuring the muscle tone according to the first embodiment of the disclosure may further include a mobile calculation device 3 (such as a notebook computer, a tablet PC, a smart phone or a cloud platform). The mobile calculation device 3 may couple with the processing end 2. The processing end 2 or the mobile calculation device 3 may execute an application to generate an spasticity level value according to the discrete-time data and two trained weighting matrixes through the use of a machine learning method. The machine learning method may be used by the medical staff (such as a therapist or a doctor) to determine the level of the dystonia of the patient. In the first embodiment, the processing end 2 may further include a communication unit 25 (such as Bluetooth, Wi-Fi or 4G-LTE) electrically connected to the processor 23. The communication unit 25 is coupled with the mobile calculation device 3. The processor 23 of the processing end 2 or the mobile calculation device 3 is able to generate the spasticity level value according to the discrete-time data and the two weighting matrixes through the use of the machine learning method (such as artificial neural network or support vector machine). For example, the outputted values of the first inertia detector 12 and the second inertia detector 22 (such as the acceleration, the magnetic gravity angle and the direction) may be used to calculate an angle value and a velocity value corresponding to the motion of the joint. Then, the spasticity level value (such as 1 to 6) may be generated based on the two weighting matrixes along with the temperature value, the strength value of the applied force, the angle value and the velocity value of the discrete-time data in each of the time points. However, this does not thus limit the disclosure. The angle value is obtained by determining an angle of posture of each of the first inertia detector 12 and the second inertia detector 22 through the coordinate transformation of the measured gravity magnetic value. The coordinate transformation includes direction cosine matrix transformation, quaternion transformation or Euler angle transformation, but is not limited thereto.

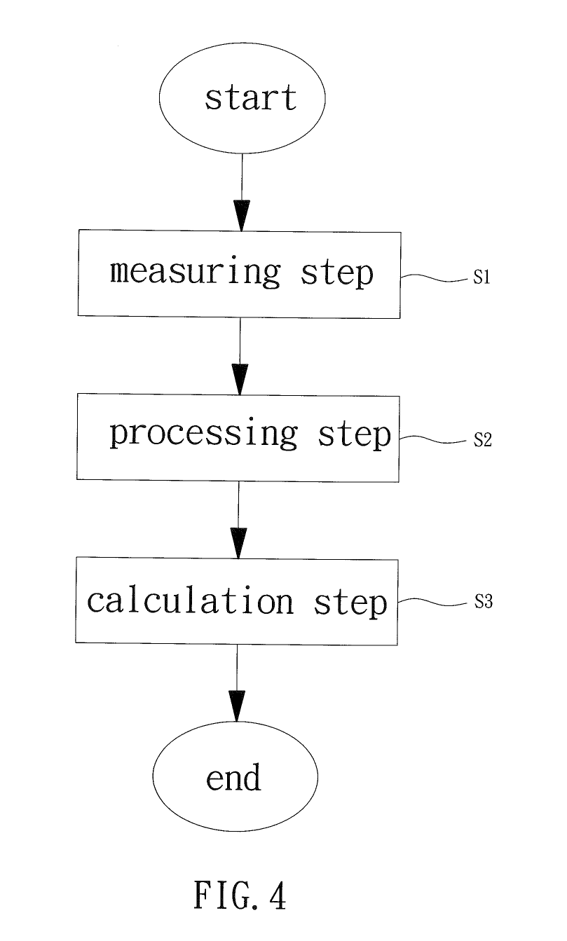

FIG. 4 shows a flowchart of a method for measuring the muscle tone according to a second embodiment of the disclosure. The method for measuring the muscle tone in the second embodiment may be used in the system of the first embodiment. The method for measuring the muscle tone includes a measuring step S1 and a processing step S2. Reference to FIGS. 1 and 2 is also needed.

In the measuring step S1, the sensing end 1 is worn on the first limb part of the limb (which is connected to the second limb part of the limb via an elbow joint as shown in FIG. 2). As such, the sensing end 1 is able to detect the information regarding the force that is applied to the first limb part and the physical quantities of the movement of the first limb part. The detections of the force that is applied to the first limb part, as well as the physical quantities of the movement of the first limb part (the 3-dimensional vector information such as the acceleration, the magnetic gravity angle and the direction), have been described in the first embodiment above.

In the processing step S2, the processing end 2 is worn on the second limb part of the limb (which is connected to the first limb part of the limb via a joint). As such, the processing end 2 is able to detect the information regarding the environment temperature and the physical quantities of the movement of the second limb part, and to collect the information regarding the movement ranges of the first and second limb parts, the temperature and the strength of the applied force according to the sampling rate during the period of measuring time. As stated above, the processing end 2 is able to detect the environment temperature and the physical quantities of the movement of the second limb part (the 3-dimensional vector information such as an acceleration, a magnetic gravity angle and a direction), and to generate the discrete-time data by collecting the information regarding the movement ranges of the first and second limb parts, the temperature and the strength of the applied force according to the sampling rate.

Referring to FIG. 4, the method for measuring the muscle tone may further include a calculation step S3. In the calculation step S3, the processing end 2 forms a discrete-time data using the collected information regarding the movement ranges of the first and second limb parts, the temperature and the strength of the applied force. Then, the processing end 2 or the mobile calculation device 3 may generate the spasticity level value according to the discrete-time data and the two trained weighting matrixes through the use of the machine learning method. As stated above, the processor 23 or the mobile calculation device 3 is able to generate the spasticity level value (levels 1-6) according to the discrete-time data and the two weighting matrixes through the use of artificial neural network or support vector machine. In the following, the learning, training and prediction of the machine are described. However, it does not thus limit the disclosure.

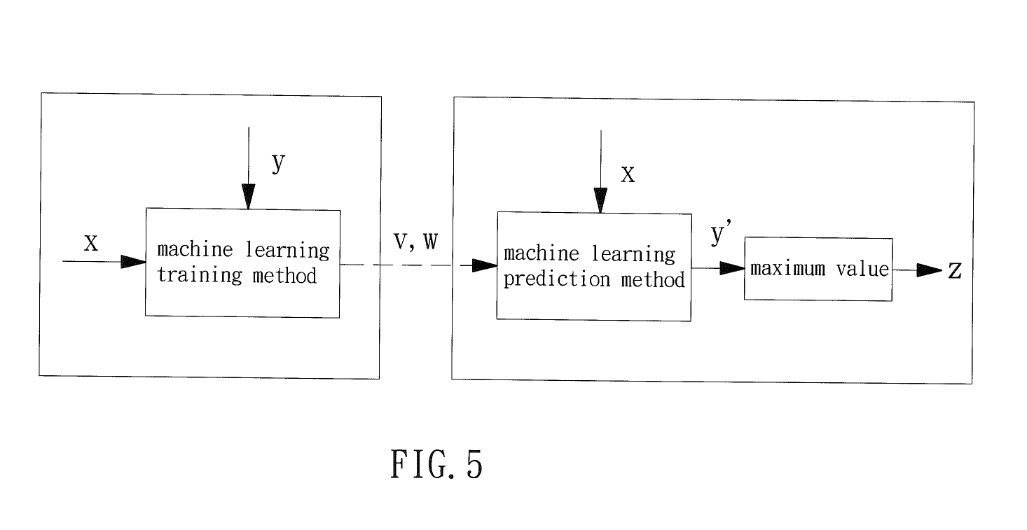

FIG. 5 shows the learning, training and prediction of the machine according to the disclosure. In the training process of the machine, the system for measuring the muscle tone in the first embodiment may be used to collect different information from different patients. Specifically, the system for measuring the muscle tone may collect the information from each of different patients regarding the angle value, the velocity value, the pressure value, the temperature value, and the predicted level determined by the doctors in each of a plurality of time points. Table 1 shows an example of the training process in which the period of measuring time is 20 seconds and the sampling rate is set as 1 time/sec.

TABLE 1
Information Regarding Angles, Accelerations,
Pressures and Temperatures at Different Time Points

| | | Velocity | | Temperature |
|---|---|---|---|---|
| Time (sec) | Angle (degree) | (cm/s) | Pressure (Kg) | (° C.) |
| 1 | 30 | 0.1 | 0 | 29.4 |
| 2 | 46 | 3.0 | 0.5 | 29.4 |
| 3 | 60 | 2.0 | 0.5 | 29.3 |
| 4 | 77 | 1.0 | 0.5 | 29.2 |
| 5 | 91 | 0.0 | 0.5 | 29.3 |
| 6 | 110 | −2.0 | 1.0 | 29.2 |
| 7 | 126 | −1.0 | 1.5 | 29.3 |
| 8 | 143 | −1.0 | 2.0 | 29.2 |
| 9 | 157 | 0.0 | 2.0 | 29.2 |
| 10 | 173 | −1.0 | 1.5 | 29.2 |
| 11 | 174 | −2.0 | 1.5 | 29.2 |
| 12 | 173 | 0.0 | 1.0 | 29.2 |
| 13 | 173 | 0.0 | 1.0 | 29.2 |
| 14 | 173 | 0.0 | 1.0 | 29.2 |
| 15 | 173 | 0.0 | 1.0 | 29.2 |
| 16 | 173 | 0.0 | 1.0 | 29.2 |
| 17 | 173 | 0.0 | 1.0 | 29.2 |
| 18 | 173 | 0.0 | 1.0 | 29.2 |
| 19 | 173 | 0.0 | 1.0 | 29.2 |
| 20 | 173 | 0.0 | 1.0 | 29.2 |

The row data or column data in Table 1 may be combined as a vector having 80 dimensions. The vector is used as the discrete-time data “x” as expressed by the formulas (1) and (1′) below.

x=[30,46,60,77, . . . ,0.1,3.0,2.0,1.0, . . . ,0,0.5,0.5,0.5, . . . ,29.4,29.4,29.3,29.2, . . . ] (1),

x=[30,0.1,0,29.4,46,3.0,0.5,29.4, . . . ,173,0.0,1.0,29.2] (1′)

TABLE 2
Ashworth Scaling

| Level of | |
|---|---|
| Spasticity | Clinical Manifestations |
| 1 | No increase in muscle tone (Normal) |
| 2 | Slight increase in muscle tone (the involved part is stuck |
| | suddenly and then becomes relaxed (or the involved part |
| | encounters a slight resistance) only when the joint is in the full |
| | range of motion during the passive flection of the involved |
| | part) |
| 3 | Slight increase in muscle tone (the involved part is stuck when |
| | the joint is in the second half of the range of motion, but |
| | encounters a slight resistance in the rest of the range of |
| | motion) |
| 4 | Moderate increase in muscle tone (the muscle tone increases |
| | in the majority of the range of motion of the joint, but the |
| | involved part is still able to move easily) |
| 5 | Significant increase in muscle tone (passive motion is |
| | difficult) |
| 6 | Muscular rigidity (the involved part is rigid and cannot move |
| | during the flection or straightening of the involved part) |

In this regard, based on the scale of the muscle tone (such as the Ashworth scale in Table 2 or the modified Ashworth scale), the doctor is able to determine that the level of spasticity “y” (as listed in Table 3 below) is 2 according to the exhibited muscle condition of the patient during the testing procedure. The result is used to generate a vector “y” having 6 dimensions (p=6), which is presented as y=[0, 1, 0, 0, 0, 0]. The testing procedure can be executed by several doctors to generate different results, and the result with the highest agreement can represent the level of spasticity “y.” Thus, the probability of misdiagnosis resulting from subject judgment of the doctor can be reduced.

TABLE 3
The determined level of Spasticity is 2

| The Level | | | | | | |
|---|---|---|---|---|---|---|
| of | | | | | | |
| Spasticity | 1 | 2 | 3 | 4 | 5 | 6 |
| y | 0 | 1 | 0 | 0 | 0 | 0 |

During the training process of the machine (artificial neural network training), a significant amount of discrete-time data “x” and spasticity levels “y” can be used. In this regard, the number of the hidden layers of the artificial neural network may be defined as “m,” which may be 0 to 1000. After training, two weighting matrixes “v” and “w” may be obtained. The weighting matrix “v” has a dimension of (n+1)×m, and the weighting matrix “w” has a dimension of (m+1)×p. For example, if m=12, the weighting matrix “v” has a dimension of 81×12, and the weighting matrix “w” has a dimension of 13×6. In each of the weighting matrixes “v” and “y,” the value of each element in the matrix is a real number between −1000 to 1000.

After the weighting matrixes “v” and “w” are trained, they may be used with the machine training method (such as artificial neural network) to predict the level of spasticity y′ of any discrete-time data. If the discrete-time data “x” of Table 1 and the weighting matrixes “v” and “w” are used, it can be obtained that the vector of the spasticity level y′ is represented as [0.513, 0.667, 0.602, 0.521, 0.379, 0.187]. The values of 0.513, 0.667, 0.602, 0.521, 0.379 and 0.187 represent the predicted values of the spasticity levels 1-6, respectively. Thus, the largest predicted value of 0.667 (which is 2) may be used as the spasticity level value (as the output “z” shown in FIG. 5). The spasticity level of 2 represents a slight increase in muscle tone (the involved part is stuck suddenly and then becomes relaxed (or the involved part encounters a slight resistance) only when the joint is in the full range of motion during the passive flection of the involved part). The result may be used by the doctor or the therapist to determine the muscle tone of the patient.

As such, the system and method for measuring the muscle tone according to the first and second embodiments of the disclosure are able to detect the strength of the force applied to the first limb part and the physical quantities of the movement of the first limb part using the sensing end 1 worn on the first limb part of the limb. Then, the processing end 2 on the second limb part of the limb is used to detect the environment temperature and the physical quantities of the movement of the second limb part, and to collect the information regarding the movement ranges of the first and second limb parts, the temperature and the strength of the applied force according to the sampling rate. As such, a discrete-time data can be generated. Finally, the discrete-time data and the two trained weighting matrixes are used to generate a spasticity level value through the use of a machine learning method. The predicted spasticity level is used by the doctor or the therapist to determine the muscle tone of the patient.

Based on the above, the system and method for measuring the muscle tone according to the first and second embodiments of the disclosure are able to generate the spasticity level value during the testing procedure of the patient. The value of the spasticity level is predicted according to the weighting matrixes that have been trained using a significant amount of training data. Thus, the accuracy of the prediction is high, and the predicted result is reliable in determining the muscle tone condition of the patient. As a result, the probability of misdiagnosis resulting from subjective judgment of the doctor is reduced. Since the system does not consist of expensive components, the manufacturing cost is low. Furthermore, the system is easy to operate so that it can be widely used in home care or medical care, increasing the objectivity of the predicted result and avoiding the misdiagnosis resulting from subjective judgment of the doctor.

Although the disclosure has been described in detail with reference to its presently preferable embodiments, it will be understood by one of ordinary skill in the art that various modifications can be made without departing from the spirit and the scope of the disclosure, as set forth in the appended claims.

What is claimed is:

1. A system for measuring a muscle tone, comprising:
a sensing end adapted to be arranged on a first limb part of a limb and comprising a pressure sensor and a first inertia detector, wherein the pressure sensor is adapted to detect a strength of a force that is applied to the first limb part of the limb, and wherein the first inertia detector is adapted to measure a plurality of physical quantities of a movement of the first limb part of the limb; and
a processing end adapted to be arranged on a second limb part of the limb, wherein the first and second limb parts are interconnected by a joint of the limb, wherein the processing end comprises a temperature sensor, a second inertia detector and a processor,
wherein the temperature sensor is adapted to detect an environment temperature, wherein the second inertia detector is adapted to measure a plurality of physical quantities of a movement of the second limb part of the limb, wherein the processor is electrically connected to the second inertia detector, the temperature sensor, the pressure sensor and the first inertia detector, and wherein the processing end is adapted to collect output signals of the temperature sensor, the pressure sensor, the first inertia detector and the second inertia detector according to a sampling rate during a period of measuring time.

2. The system for measuring the muscle tone as claimed in claim 1, wherein the processing end is adapted to generate a discrete-time data from the output signals of the temperature sensor, the pressure sensor, the first inertia detector and the second inertia detector, and to generate a spasticity level value according to the discrete-time data and two trained weighting matrixes through the use of a machine learning method.

3. The system for measuring the muscle tone as claimed in claim 2, wherein the processing end is adapted to calculate an angle value and a velocity value corresponding to a motion of the joint according to the output signals of the first and second inertia detectors, and to generate the spasticity level value according to the two trained weighting matrixes and the environment temperature, the strength of the force, the angle value and the velocity value in each of a plurality of time points.

4. The system for measuring the muscle tone as claimed in claim 1, wherein the processing end further comprises a communication unit electrically connected to the processor and coupled with a mobile calculation device, wherein the processing end is adapted to generate a discrete-time data from the output signals of the temperature sensor, the pressure sensor, the first inertia detector and the second inertia detector, and to generate a spasticity level value according to the discrete-time data and two trained weighting matrixes through the use of a machine learning method.

5. The system for measuring the muscle tone as claimed in claim 4, wherein the mobile calculation device is adapted to generate an angle value and a velocity value corresponding to a motion of the joint according to the output signals of the first and second inertia detectors, and to generate the spasticity level value according to the two trained weighting matrixes and the environment temperature, the strength of the force, the angle value and the velocity value in each of a plurality of time points.

6. The system for measuring the muscle tone as claimed in claim 2, wherein the machine learning method is artificial neural network or support vector machine.

7. The system for measuring the muscle tone as claimed in claim 4, wherein the machine learning method is artificial neural network or support vector machine.

8. The system for measuring the muscle tone as claimed in claim 1, wherein each of the first and second inertia detectors comprises a three-axis accelerometer, a three-axis gyroscope and a three-axis magnetometer.

9. The system for measuring the muscle tone as claimed in claim 1, wherein the processing end comprises a display electrically connected to the processor.

10. A method for measuring a muscle tone for use in a muscle tone measuring system, wherein the muscle tone measuring system comprises a sensing end and a processing end, wherein the processing end is electrically connected to the sensing end and coupled with a mobile calculation device, the method comprises:
arranging the sensing end on a first limb part of a limb, so that the sensing end is able to detect a strength of a force that is applied to the first limb part of the limb and a plurality of physical quantities of a movement of the first limb part of the limb; and
arranging the processing end on a second limb part of the limb, so that the processing end is able to detect an environment temperature and a plurality of physical quantities of a movement of the second limb part, and to generate a discrete-time data by collecting the plurality of physical quantities of the movement of the first limb part, the plurality of physical quantities of the movement of the second limb, the environment temperature and the strength of the force according to a sampling rate during a period of measuring time, wherein the first and second limb parts are interconnected by a joint of the limb.

11. The method for measuring the muscle tone as claimed in claim 10, wherein the processing end is adapted to generate a discrete-time data from the plurality of physical quantities of the movement of the first limb part, the plurality of physical quantities of the movement of the second limb, the environment temperature and the strength of the force, and to generate a spasticity level value according to the discrete-time data and two trained weighting matrixes through the use of a machine learning method.

12. The method for measuring the muscle tone as claimed in claim 11, wherein the processing end is adapted to calculate an angle value and a velocity value corresponding to a motion of the joint according to the plurality of physical quantities of the movement of the first limb part and the plurality of physical quantities of the movement of the second limb, and to generate the spasticity level value according to the two trained weighting matrixes and the environment temperature, the strength of the force, the angle value and the velocity value in each of a plurality of time points.

13. The method for measuring the muscle tone as claimed in claim 10, wherein the processing end is adapted to generate a discrete-time data from the plurality of physical quantities of the movement of the first limb part, the plurality of physical quantities of the movement of the second limb, the environment temperature and the strength of the force, and the mobile calculation device is adapted to generate a spasticity level value according to the discrete-time data and two trained weighting matrixes through the use of a machine learning method.

14. The method for measuring the muscle tone as claimed in claim 13, wherein the mobile calculation device is adapted to calculate an angle value and a velocity value corresponding to a motion of the joint according to the plurality of physical quantities of the movement of the first limb part and the plurality of physical quantities of the movement of the second limb, and to generate the spasticity level value according to the two trained weighting matrixes and the environment temperature, the strength of the force, the angle value and the velocity value in each of a plurality of time points.

15. The method for measuring the muscle tone as claimed in claim 11, wherein the machine learning method is artificial neural network or support vector machine.

16. The method for measuring the muscle tone as claimed in claim 13, wherein the machine learning method is artificial neural network or support vector machine.
